(19) United States
(12) Patent Application Publication
(10) Pub. No.: US 2025/0161391 A1
(43) Pub. Date: May 22, 2025

(54) UROLITHIN COMBINATION

(71) Applicant: Amazentis SA, Lausanne (CH)

(72) Inventors: Christopher L. Rinsch, Lausanne (CH); Anurag Singh, Lausanne (CH); Davide D'Amico, Renens (CH)

(21) Appl. No.: 18/925,886

(22) Filed: Oct. 24, 2024

(30) Foreign Application Priority Data
Oct. 24, 2023 (GB) 2316295.1

(51) Int. Cl. A61K 36/258; A61K 9/06; A61K 9/16; A61K 31/352; A61K 31/704; A61K 45/06; A61P 29/00; A61P 37/06

(52) U.S. Cl. CPC A61K 36/258; A61K 9/06; A61K 9/1611; A61K 9/1623; A61K 9/1658; A61K 31/352; A61K 31/704; A61K 45/06; A61P 29/00; A61P 37/06

(57) ABSTRACT

The invention relates to compositions comprising urolithins with active agents, particularly compositions comprising urolithins and one or more ginseng-derived compounds or a ginseng extract; and/or one of more notoginseng-derived compounds or a notoginseng extract. The invention also relates to uses of such compositions, for use for the treatment or prevention of diseases, disorders and conditions, and for non-therapeutic uses.

### RELATED APPLICATION

This application claims the benefit of priority to UK Patent Application No. GB 2316295.1, filed Oct. 24, 2023.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO 2012/088519 (Amazentis SA). In WO 2007/127263 (The Regents of the University of California), the use of urolithins for the treatment of various neoplastic diseases is described.

International patent publication WO 2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, al-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder. Administration may also be to generally healthy subjects for boosting general health, for example, muscle performance, immune health and or brain function.

Ginseng is an ancient perennial herb belonging to the family Araliaceae and genus Panax (Panax ginseng) which has been used for medical therapeutics for thousands of years, particularly in China and other Asian cultures. In the last three decades, it has become one of the most popular herbs worldwide. It is used in agricultural products, dietary and health supplements, and medicines in different countries. The dried roots and rhizomes of ginseng contain many physiologically important constituents. These include ginseng saponins, ginseng oils and phytosterol, carbohydrates and sugars, organic acids, nitrogenous substances, amino acids and peptides, vitamins and minerals, and certain enzymes that have been isolated and characterized. The saponins are referred to as ginsenosides and are the most active constituents. Although the effects of ginseng are not solely dependent on ginsenosides, and other active ingredients have been described, such as gintonins.

The ginsenosides are triterpene saponins and to date, nearly 200 ginsenosides have been reported; some of these, such as Rb1, Rb2, Rc, Rd, Re, and Rg1, are considered major ginsenosides. These compounds have multifaceted pharmacological activities because of their steroidal structure. They can interact with membrane-bound ion channels, cell membranes, and extracellular and intracellular receptors, and as a result cause alterations at the transcriptional level. They show various anti-inflammatory, antioxidant, antibacterial, antiviral, and antifungal activities. Moreover, they have been demonstrated to have therapeutic potential in hypertension, stress, and different neurological disorders such as Alzheimer's disease (AD), Parkinson disease (PD), and Huntington disease. For a review of ginsenosides see Ratan et al (2021) Journal of Ginseng Research 45(2), 199-210.

Notoginseng is a related perennial herb, also belonging to the family Araliaceae and genus Panax (Panax notoginseng) and is also widely used in traditional Chinese medicine. Notoginseng also contains saponins which are referred to as notoginsenosides, although some of the notoginsenosides are also found in ginseng and some are unique to notoginseng. A review of ginsenosides and notoginsenosides is Liu et al (2020) Pharmacological Research 161, 105263.

### SUMMARY

The invention relates to compositions comprising urolithins with active agents, particularly compositions comprising urolithins and one or more ginseng-derived compounds or a ginseng extract; and/or one of more notoginseng-derived compounds or a notoginseng extract. The invention also relates to uses of such compositions, for use for the treatment or prevention of diseases, disorders and conditions, and for non-therapeutic uses.

Surprisingly, it was found that combining the components of ginseng and/or notoginseng with urolithins, such urolithin A is particularly effective, especially for enhancing mitochondrial function and reducing inflammatory and oxidative stress. It is believed that urolithins and the components of ginseng and/or notoginseng modulate different biochemical pathways leading to synergistic combinations of the compounds.

One aspect of the invention provides a composition comprising:
(a) a compound of formula (I), or a salt, prodrug, metabolite or derivative thereof;

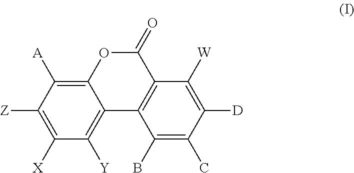

wherein:
A, B, C, D, W, X, Y and Z are each independently selected from H and OH; and
(b) one or more ginseng-derived compounds or a ginseng extract; and/or one of more notoginseng-derived compounds or a notoginseng extract.

### BRIEF DESCRIPTION OF THE FIGURES

FIG. 1: IL-6 concentration following treatment with UA or Rg1 at the indicated doses.

FIG. 2: IL-6 concentration following treatment with UA or Rg1, or a combination of UA and Rg1, at the indicated doses.

FIG. 3: IL-6 concentration following treatment with UA or Rg1, or a combination of UA and Rg1, at the indicated doses.

### DETAILED DESCRIPTION

According to the first embodiment of the invention there is provided a composition comprising:
(a) a compound of formula (I), or a salt, prodrug, metabolite or derivative thereof;

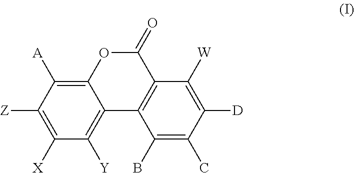

wherein:
   A, B, C, D, W, X, Y and Z are each independently selected from H and OH; and
(b) one or more ginseng-derived compounds or a ginseng extract; and/or one of more notoginseng-derived compounds or a notoginseng extract.

In a further embodiment of the invention, there is provides a composition comprising:
(a) a compound of formula (I), or a salt, prodrug, metabolite or derivative thereof; and
(b) one or more ginseng-derived compounds or a ginseng extract.

In a further embodiment of the invention, there is provides a composition comprising:
(a) a compound of formula (I), or a salt, prodrug, metabolite or derivative thereof; and
(b) one of more notoginseng-derived compounds or a notoginseng extract.

The most frequently used part of the ginseng plant for traditional therapeutic applications is the root (rhizome and/or radix) and the two most common post-harvest processes for the processing of ginseng, to facilitate long-term storage, are drying (white ginseng) and steaming (red ginseng). Processing does affect the ginsenoside composition of white and red ginseng. For a review, see He et al (2018) Planta Med. 84,845-854. Therefore, according to a further aspect of the invention, there is provided a composition wherein the ginseng-derived compounds or ginseng extract is derived from red ginseng and/or wherein the ginseng-derived compounds or ginseng extract is derived from white ginseng. In a further embodiment, ginseng-derived compounds or ginseng extract is derived from fermented red ginseng and/or derived from fermented white ginseng.

Ginseng and notoginseng comprise a number of constituents, including saponins (ginsenosides and notoginsenosides), polysaccharides, amino acids, volatile oils, and polyacetylenes.

Ginsenosides are believed to be primarily responsible for the actions of ginseng, and notoginsenosides are believed to be primarily responsible for the actions of notoginseng.

Therefore, according to a further embodiment of the invention there is provided a composition wherein:
(a) the ginseng-derived compound comprises one or more ginsenosides; and/or
(b) the notoginseng-derived compound comprises one or more notoginsenosides.

Ginsenosides and notoginsenosides are triterpenoidal glycosides with a high chemical variation, depending on the linkage position and number of sugars on the aglycone skeleton and can be classified as dammarane-type saponins, (for example, protopanaxadiol-type saponins and protopanaxatriol-type saponins), ocotillol-type saponins, or oleanane-type sapoinins. Therefore, according to a further aspect of the invention, there is provided a composition wherein the ginsenosides comprise dammarane-type saponins, (for example, protopanaxadiol-type saponins and protopanaxatriol-type saponins), ocotillol-type saponins, and/or oleanane-type sapoinins.

Notoginseng contains only dammarane-type saponins (for example, protopanaxadiol-type saponins and protopanaxatriol-type saponins), and ocotillol-type saponins. Therefore, according to a further embodiment of the invention, there is provided a composition wherein the notoginsenosides comprise dammarane-type saponins, (for example, protopanaxadiol-type saponins and protopanaxatriol-type saponins) and/or ocotillol-type saponins.

A number of ginsenosides and notoginsenosides have been identified. Some of these saponin are shared between ginseng and notoginseng, for example, Rb1, Rd, Re, Rg1, Rg2, Rh1, and gypenoside XVII. Therefore, according to a further aspect of the invention, there is provided a composition of the invention, wherein the ginsenosides and/or notoginsenosides comprise one or more selected from Rb1, Rd, Re, Rg1, Rg2, Rh1, and gypenoside XVII. Of these Rg1 and Rb1 are the most abundant and therefore, according to a further embodiment of the invention, there is provided a composition wherein the ginsenosides and/or notoginsenosides comprise one or more selected from Rg1 and Rb1.

According to a further embodiment of the invention, there is provided a composition of the invention wherein the ginsenosides are one or more selected from Rb1, Rb2, Rg3, Rh2, Rh3, Rg1, Rg2, and Rh1.

According to a further embodiment of the invention, there is provided a composition of the invention comprising urolithin A, or salt thereof, and ginsenoside Rg1.

According to a further embodiment of the invention, there is provided a composition of the invention comprising urolithin A, or salt thereof, and ginsenoside Rg1 and ginsenoside Rb1.

Notoginseng also contains notoginsenosides which are different from ginsenoside compounds found in ginseng, these include: R1, Rt, R2, R3, R4, and R6. Therefore, according to a further embodiment of the invention, there is provided a composition of the invention wherein the notoginsenosides are one or more selected from R1, Rt, R2, R3, R4, and R6.

The effects of ginseng are not fully explained by ginsenosides. More recently, another class of active ingredients called gintonin has been identified. Gintonin is a complex of glycosylated ginseng proteins containing lysophosphatidic acids (LPAs). Therefore, according to a further embodiment of the invention there is provided a composition of the invention wherein the ginseng-derived compound comprises one or more gintonins.

### Ginseng Extracts

Generally, a ginseng extract may be prepared from ginseng leaves and/or roots (preferably roots), in a variety of ways known in the art (see, for example, Oshima et al. J. Nat. Prod., March-April 1987, pp. 188-190; Shoji, J. Chemistry of Ginseng; in Yakuyoninjin (Recent-Studies on Ginseng, Tokyo, Kyoritsu Publishing Co., 1981, p. 10), and normally yields 10-40% by weight of the ginseng plant. Methods for preparing ginseng extracts are also disclosed in International application, WO 1999/030725 (PCT/US98/25724).

### Compounds of Formula (I) (Urolithins)

Compounds of formula (I) (Urolithins) are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

| (I) | | | | | | |
|---|---|---|---|---|---|---|
| 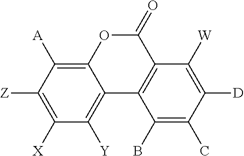 | | | | | | |
| | Substituent of structure (I) | | | | | |
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OF |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the combinations of the invention.

In one aspect of a combinations of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

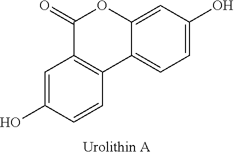 [SMILES: O=c1oc2cc(O)ccc2c2ccc(O)cc12]

According to one embodiment there is provided a combination, composition, use or method of the invention wherein the compound of formula (I) is urolithin A.

According to one embodiment there is provided a combination, composition, use or method of the invention wherein the compound of formula (I) is urolithin B.

According to one embodiment there is provided a combination, composition, use or method of the invention wherein the compound of formula (I) is urolithin C.

According to one embodiment there is provided a combination, composition, use or method of the invention wherein the compound of formula (I) is urolithin D.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

### Urolithin Administration/Dosage Regimes

Compositions of the invention may be administered by any suitable method, for example, orally or topically.

### Oral Administration

In one embodiment, the administration of compositions of the present invention involves oral administration of a urolithin of formula (I) or salt thereof to a subject in a daily amount in the range of about 1.1 to about 8.8 mmol, for example, from about 1.7 to about 6.0 mmol per day, for example, from about 1.7 to about 2.7 mmol per day, or from about 2.8 to about 6.0 mmol per day. As discussed below, administration is preferred in the range 125 mg to 200 mg of a compound of Formula (1), for example, urolithin A, for example, 250 mg to 2000 mg urolithin A (which corresponds to about 1.1 to 8.8 mmol), for example 250 mg to 1500 mg, such as 250 mg to 1000 mg, which results in a surprisingly good pharmacokinetic profile. In one embodiment the dose is 125 mg/day, in a alternative embodiment 250 mg/day, in an alternative embodiment the dose is 500 mg/day and in another embodiment the dose is 1000 mg/day. In a further embodiment, the dose is 1500 mg/day. In a further embodiment, the dose is 2000 mg/day.

In a further embodiment, administration doses are selected from:
125 mg once or twice a day;
250 mg once or twice a day;
500 mg once or twice a day;
750 mg once or twice a day;
1000 mg once or twice a day;
1250 mg once or twice a day; or
1500 mg once or twice a day

The uses and methods of the present invention involve daily administration of the compound of formula (I) or salt thereof, or of a composition comprising the compound or salt. In some embodiments, the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

The uses or methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In some preferred embodiments the uses or method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the use or method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from about 100 mg to about 600 mg/day for example, from about 125 mg to about 600 mg/day for example, about 200 mg to about 600 mg per day, for example from about 300 mg to about 600 mg/day from 400 to 600 mg/day or an amount of urolithin A in said ranges twice per day. In a preferred embodiment the method involves administration of urolithin A in an amount in the range of from 450 to 550 mg, more preferably approximately 500 mg per day or twice per day. In a further preferred range, the method involves administration of urolithin A in the range from about 200 mg to about 300 mg, more preferably about 250 mg per day or twice per day. In a yet further preferred range, the method involves administration of urolithin A in the range from about 100 mg to about 150 mg, more preferably about 125 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the uses or methods involve administering urolithin A to the subject in an amount in the range of from 4.5 to 11 mg/kg/day, such as 4.5 to 8.5 mg/kg/day. In another embodiment, the uses or methods involve administering urolithin A to the subject in an amount in the range of 5 to 9 mg/kg/day. In another embodiment, the uses or methods involve administering urolithin A to the subject in an amount in the range of from 6.0 to 8 mg/kg/day.

In other preferred embodiments, the uses or methods involve administering urolithin A to the subject in an amount in the range of from 9 to 18 mg/kg/day such as 9 to 17 mg/kg/day. In another embodiment, the uses or methods involve administering urolithin A to the subject in an amount in the range of from 10 to 17 mg/kg/day. In another embodiment, the uses or methods involve administering urolithin A to the subject in an amount in the range of from 11 to 16 mg/kg/day.

Dosage regimes which combine two or more of a 125 mg, a 250 mg, a 500 mg dose and a 1000 mg dose may be advantageous. For example, a twice daily dosage regime which combines a first dose of 1000 mg and a second dose several hours later of 500 mg or a first dose of 500 mg and a second dose hours later of a 250 mg dose, or a 250 mg and a second dose hours later of a 125 mg dose. Said 125 mg, 250 mg or 500 mg dose may be 6-18 hours after the 250 mg, 500 mg or 1000 mg dose, for example 8-12 hours after the 250 mg, 500 mg or 1000 mg dose. For example, about 12 hours after the 250 mg, 500 mg or 1000 mg dose. Thus, according to a further aspect of the invention there is provided the treatment of a disease, disorder or condition with a compound of Formula (I) which comprises a twice daily dosage regime comprising a first dose of 250 mg or 500 mg or 1000 mg, followed by a second dose of 125 mg or 250 mg or 500 mg wherein the two doses are separated by 6-18 hours.

The compound of formula (I) or salt thereof, or composition containing the compound of salt, may be administered at any suitable time, for example, it may be administered in the morning after sleep or in the evening. In some embodiments, it may be preferable for the method to be performed at approximately the same time(s) each day, for example within 15, 30, 60 or 120 minutes of a given time point.

In a further embodiment, there is provided a composition wherein the compound of formula (I) is administered at a dose of about 4.5-18 mg/kg.

### Topical Administration

In a further embodiment of the invention, when the administration is topical administration, there is provided a composition of the invention, comprising:
(a) about 0.1% (w/w) to about 5% (w/w) of a compound of formula (I), or a salt thereof, for example, urolithin A, or a salt thereof; and
(b) about 5% (w/w) to about 30% (w/w) of a ginseng or notoginseng extract.

In a further embodiment of the invention, when the administration is topical administration, there is provided a composition of the invention, comprising:
(a) about 0.1% (w/w) to about 5% (w/w) of a compound of formula (I), or a salt thereof, for example, urolithin A, or a salt thereof; and
(b) about 0.1% (w/w) to about 10%, for example, about 0.1% to about 7%, about 0.1% (w/w) to about 5% (w/w) of one or more ginsenosides and/or notoginsenosdies.

In a further embodiment the concentration range of a compound of formula (I), or a salt thereof, for example, urolithin A, or a salt thereof, for topical administration is from about 0.8% (w/w) to about 4% (w/w) of the composition, for example, about 0.8% (w/w) to about 3% (w/w) of the composition, for example, about 0.8% (w/w) to about 2% w/w of the composition, for example about 1% (w/w) to 2% w/w of the composition, for example about 1% (w/w) to about 1.5% w/w, for example about 1% w/w, such as about 0.8% (w/w), or about 0.9% (w/w), or about 1% (w/w), or about 1.5% (w/w) or about 2% (w/w) or about 2.5% (w/w) or about 3% (w/w) or about 4% (w/w) of the composition. In a further embodiment of the invention, the concentration range of a compound of formula (I), or a salt thereof, for example, urolithin A, or salt thereof, is about 1% (w/w) to about 5% w/w, for example, about 2% (w/w) to about 5% w/w, for example, about 3% (w/w) to about 5% w/w, such as about 4% (w/w) to about 5% w/w.

In a further embodiment, the concentration range of the ginsenocides and/or notoginsenosides, for example, Rg1, is about 0.1% to about 7%.

### Ginsenosides and Notoginsenosides Administration/Dosage Regimes

Ginsenosides and notoginsenosides comprises ginsenosides and notoginsenosides, and any of its salts, metabolites and/or its derivatives that have the same or an equivalent biological functionality. Reference to the integers ginsenosides and notoginsenosides also refer to prodrugs of ginsenosides and notoginsenosides.

Compositions of the invention comprise between 500 mg to 3000 mg of a ginseng and/or notoginseng extract. Compositions of the invention comprise between about 1 mg to about 50 mg ginsenosides and/or notoginsenosides

In a further embodiment there is provided a composition comprising:
(a) 100 to 2000 mg of a compound of formula (I), or a salt, prodrug, metabolite or derivative thereof, for example, urolithin A; and
(b) about 100 mg to about 1000 mg of a notoginseng extract.

In a further embodiment there is provided a composition comprising:
(a) 100 to 2000 mg of a compound of formula (I), or a salt, prodrug, metabolite or derivative thereof, for example, urolithin A; and
(b) about 100 mg to about 1000 mg of a notoginseng extract.

In a further embodiment there is provided a composition comprising:
(a) 100 to 2000 mg of a compound of formula (I), or a salt, prodrug, metabolite or derivative thereof, for example, urolithin A; and
(b) about 100 mg to about 1000 mg of a mixture of a ginseng and a notoginseng extract.

In a further embodiment there is provided a composition comprising:
(a) 100 to 2000 mg of a compound of formula (I), or a salt, prodrug, metabolite or derivative thereof, for example, urolithin A; and
(b) about 0.5 mg to about 50 mg ginsenosides, for example, about 1 mg to about 50 mg ginsenosides, about 3 mg to about 40 mg, about 10 mg to about 40 mg, about 10 mg to about 30 mg, about 1 mg, about 5 mg, about 10 mg, about 20 mg, about 30 mg, about 40 mg or about 50 mg.

In a further embodiment there is provided a composition comprising:
(a) 100 to 2000 mg of a compound of formula (I), or a salt, prodrug, metabolite or derivative thereof, for example, urolithin A; and
(b) about 0.5 mg to about 50 mg notoginsenosides, for example, about 1 mg to about 50 mg notoginsenosides, about 3 mg to about 40 mg, about 10 mg to about 40 mg, about 10 mg to about 30 mg, about 1 mg, about 5 mg, about 10 mg, about 20 mg, about 30 mg, about 40 mg or about 50 mg.

In a further embodiment there is provided a composition comprising:
(a) 100 to 2000 mg of a compound of formula (I), or a salt, prodrug, metabolite or derivative thereof, for example, urolithin A; and
(b) about 0.5 mg to about 50 mg of a mixture or one of more ginsenosides and one or more notoginsenosides, for example, about 1 mg to about 50 mg, about 3 mg to about 40 mg, about 10 mg to about 40 mg, about 10 mg to about 30 mg, about 1 mg, about 5 mg, about 10 mg, about 20 mg, about 30 mg, about 40 mg or about 50 mg.

In a further embodiment, there is provided a composition, comprising:
(a) about 0.25% (w/w) to about 5% of a compound of formula (I); or a salt thereof, for example, urolithin A, or salt thereof, and
(b) about 10% (w/w) to about 50% (w/w) of a ginseng extract, for example, about 20% (w/w) to about 40% (w/w), about 10% (w/w), about 20% (w/w), about 30% (w/w), about 40% w/w) or about 50% (w/w).

In a further embodiment, there is provided a composition, comprising:
(a) about 0.25% (w/w) to about 5% of a compound of formula (I); or a salt thereof, for example, urolithin A, or salt thereof, and
(b) about 10% (w/w) to about 50% (w/w) of a notoginseng extract, for example, about 20% (w/w) to about 40% (w/w), about 10% (w/w), about 20% (w/w), about 30% (w/w), about 40% w/w) or about 50% (w/w).

In a further embodiment, there is provided a composition, comprising:
(a) about 0.25% (w/w) to about 5% of a compound of formula (I); or a salt thereof, for example, urolithin A, or salt thereof, and
(b) about 10% (w/w) to about 50% (w/w) of a mixture of a ginseng extract and a notoginseng extract, for example, about 20% (w/w) to about 40% (w/w), about 10% (w/w), about 20% (w/w), about 30% (w/w), about 40% w/w) or about 50% (w/w).

In a further embodiment, there is provided a composition, comprising:
(a) about 0.25% (w/w) to about 5% of a compound of formula (I); or a salt thereof, for example, urolithin A, or salt thereof; and
(b) about 1% (w/w) to about 10% (w/w) of one or more ginsenosides, for example, about 3% (w/w) to about 8% (w/w), for example, about 4% (w/w) to about 6% (w/w), for example, about 4% (w/w), about 5% (w/w) or about 6% (w/w).

In a further embodiment, there is provided a composition, comprising:
(a) about 0.25% (w/w) to about 5% of a compound of formula (1); or a salt thereof, for example, urolithin A, or salt thereof; and
(b) about 1% (w/w) to about 10% (w/w) of one or more notoginsenosides, for example, about 3% (w/w) to about 8% (w/w), for example, about 4% (w/w) to about 6% (w/w), for example, about 4% (w/w), about 5% (w/w) or about 6% (w/w).

In a further embodiment, there is provided a composition, comprising:
(a) about 0.25% (w/w) to about 5% of a compound of formula (1); or a salt thereof, for example, urolithin A, or salt thereof; and
(b) about 1% (w/w) to about 10% (w/w) of a mixture of one or more ginsenosides, and one or more notoginsenosides, for example, about 3% (w/w) to about 8% (w/w), for example, about 4% (w/w) to about 6% (w/w), for example, about 4% (w/w), about 5% (w/w) or about 6% (w/w).

### Additional/Combination Therapy

Compositions of the invention may comprise one of more further active agents, for use in the treatment or prevention of diseases, disorders or conditions.

Any active agent which is known to be useful, or which has been used or is currently being used for the treatment or prevention of diseases, disorders or conditions can be used with a combination of the invention. See, e.g., Gilman et al, Goodman and Gilman's: The Pharmacological Basis of Therapeutics, 13th ed., McGraw-Hill, New York, 2017; The Merck Manual of Diagnosis and Therapy, Robert S. Porter, M.D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians' Desk Reference (71st ed. 2016) for information regarding therapies (e.g., prophylactic or therapeutic agents) which have been or are currently being used for the treatment or prevention of diseases, disorders or conditions associated with hair, nails and/or skin.

Compositions of the invention may also comprise an autophagy inducer and/or a mitochondrial biogenesis promoting agent. Therefore, according to a further aspect of the invention there is provided a composition of the invention, further comprising an autophagy inducer and/or a mitochondrial biogenesis promoting agent.

Examples of autophagy inducers include, but not limited to, caffeine, omega-3 polyunsaturated fatty acids, resveratrol, spermidine, vitamin D, pterostilbene, fistein, genistein, quercetin, apigenin, kaempferol, minoxidil, actinonin, and trehalose.

Examples of mitochondrial biogenesis promoting agents include, but are not limited to, PPAR-PGC-1α axis activators ( ), AMPK activators (for example, resveratrol), Sirti agonists (for example, quercetin, resveratrol), anti-oxidants (such as L-carnitine, coenzyme Q₁₀, MitoQ₁₀ and other mitochondria-targeted antioxidants, N-acetylcysteine (NAC), vitamin C, vitamin E vitamin K1, vitamin B, sodium pyruvate and α-lipoic acid) and NAD precursors (for example, niacinamide).

In a further embodiment of the invention, there is provided a composition comprising:
(a) a compound of formula (I), or a salt thereof, for example, a urolithin A or a salt thereof;
(b) a ginseng extract and/or a notoginseng extract; and
(c) co-enzyme Q₁₀.

In a further embodiment of the invention, there is provided a composition comprising:
(a) a compound of formula (I), or a salt thereof, for example, a urolithin A or a salt thereof;
(b) one or more ginsenocides and/or one or more notoginsenosides; and
(c) co-enzyme Q₁₀.

In a further embodiment of the invention, there is provided a composition comprising:
(a) a compound of formula (I), or a salt thereof, for example, a urolithin A or a salt thereof;
(b) a ginseng extract and or a notoginseng extract; and
(c) an NAD precursors (for example, niacinamide).

In a further embodiment of the invention, there is provided a composition comprising:
(a) a compound of formula (I), or a salt thereof, for example, a urolithin A or a salt thereof;
(b) one or more ginsenocides and/or one or more notoginsenosides; and
(c) an NAD precursors (for example, niacinamide).

### Uses

Compositions of the invention can be used for both therapeutic and non-therapeutic uses, finding use in the treatment of various diseases as well as health conditions not considered to be a disease. In particular, disease and non-disease health conditions may be characterised by an inadequate mitochondrial activity. Therefore, according to one embodiment of the invention there is provided a composition of the invention for use as a medicament for the treatment of a disease disorder or condition. According to a further embodiment of the invention there is provided a non-therapeutic method of use of a composition of the invention.

The compositions of the invention can be used as a dietary supplement, as a functional food and as a medical food.

Compositions of the invention find utility in the treatment of disease, disorders and conditions. Therefore, according to a further aspect of the invention, there is provided a composition, comprising:
(a) a compound of formula (I), or a salt, prodrug, metabolite or derivative thereof; and
(b) one or more ginseng-derived compounds or a ginseng extract; and/or one of more notoginseng-derived compounds or a notoginseng extract;
for use as a medicament for the treatment of a disease disorder or condition.

According to a further aspect of the invention there is provided a composition, comprising
(a) a compound of formula (I), or a salt, prodrug, metabolite or derivative thereof, for example, urolithin A; and
(b) one or more ginseng-derived compounds or a ginseng extract; and/or one of more notoginseng-derived compounds or a notoginseng extract;
for use in the treatment, prevention or management of a mitochondria-related condition associated with altered mitochondrial function or reduced mitochondrial density.

According to a further aspect of the invention there is provided a non-therapeutic method for preventing or managing a mitochondria-related condition associated with altered mitochondrial function or reduced mitochondrial density, comprising administration of an effect amount of a composition comprising:
(a) a compound of formula (I), or a salt, prodrug, metabolite or derivative thereof, for example, urolithin A; and
(b) one or more ginseng-derived compounds or a ginseng extract; and/or one of more notoginseng-derived compounds or a notoginseng extract.

According to a further aspect of the invention there is provided a combination or composition, comprising
(a) a compound of formula (I), or a salt, prodrug, metabolite or derivative thereof, for example, urolithin A; and
(b) one or more ginseng-derived compounds or a ginseng extract; and/or one of more notoginseng-derived compounds or a notoginseng extract;
for use in the treatment, prevention or management of a mitochondria-related disease associated with altered mitochondrial function or reduced mitochondrial density.

According to a further aspect of the invention there is provided a composition, comprising
(a) a compound of formula (I), or a salt, prodrug, metabolite or derivative thereof, wherein the compound of formula (I) is a urolithin, for example, urolithin A, urolithin B, urolithin C or urolithin D, such as urolithin A; and;
(b) one or more ginseng-derived compounds or a ginseng extract; and/or one of more notoginseng-derived compounds or a notoginseng extract;
for use in the treatment, prevention or management of a mitochondria-related disease.

According to a further aspect of the invention there is provided a combination or composition, comprising
(a) a compound of formula (I), or a salt, prodrug, metabolite or derivative thereof, wherein the compound of formula (I) is a urolithin, for example, urolithin A, urolithin B, urolithin C or urolithin D, such as urolithin A; and;
(b) one or more ginseng-derived compounds or a ginseng extract; and/or one of more notoginseng-derived compounds or a notoginseng extract;
for use in increasing or maintaining mitochondrial function.

According to a further aspect of the invention there is provided a non-therapeutic method of increasing or maintaining mitochondrial function, comprising administration of an effect amount of a composition comprising:
(a) a compound of formula (I), wherein the compound of formula (I) is a urolithin, for example, urolithin A, urolithin B, urolithin C or urolithin D, such as urolithin A; and;
(b) one or more ginseng-derived compounds or a ginseng extract; and/or one of more notoginseng-derived compounds or a notoginseng extract.

### Therapeutic Uses

### Inflammatory Diseases, Disorders and Conditions

According to a further embodiment of the invention, there is provided a composition of the invention for inhibiting inflammation. According to a further embodiment of the invention, there is provided a composition of the invention for relieving or treating acute inflammation or chronic inflammation-induced diseases. In one embodiment, the acute inflammation is, for example, influenza virus pneumonia, new coronavirus pneumonia, and acute lung injury inflammation. In a further embodiment, the chronic inflammation is, for example, chronic obstructive pneumonia, chronic asthmatic airway inflammation and chronic obstructive pulmonary disease (COPD).

According to a further embodiment of the invention, there is provided a composition of the invention, for treating or preventing an inflammatory disease, for example, an inflammatory disease selected from inflammatory skin diseases, Crohn's disease, ulcerative colitis, peritonitis, osteomyelitis, meningitis, meningitis, encephalitis, pancreatitis, trauma-induced shock, bronchial asthma, allergic rhinitis, cystic fibrosis, Inflammatory bowel disease, acute bronchitis, chronic bronchitis, osteoarthritis, gout, spondyloarthropathies, ankylosing spondylitis, intestinal spondylitis, inflammatory arthropathy, ankylosing spondylitis, reactive arthropathy, infectious arthritis, systemic lupus erythematosus, recurrent and psoriasis. For example, an inflammatory disease selected from arthritis, gonococcal arthritis, tuberculous arthritis, viral arthritis, fungal arthritis, rheumatoid arthritis, rheumatoid polyposis muscle pain, arthritic arthritis, calcium arthritis, non-articular rheumatism, bursitis, and hay fever.

### Infectious Diseases

According to one embodiment of the invention, there is provided a composition of the invention for use in the treatment of microbial diseases. In a further embodiment, there is provided a composition of the invention for use in the treatment of viral diseases.

According to a future embodiment of the invention, there is provided a composition of the invention, for use in treating a post-viral illness, for example, symptoms associated with recovery from the viral disease or infection in the subject. Symptoms associated with recovery from a viral disease comprise fatigue, post-exertional malaise (PEM), problems with memory or concentration, sore throat, headache, muscle or joint pain, dizziness, brain fog, shortness of breath, and/or unrefreshing sleep. In one embodiment, the post-viral illness is selected from myalgic encephalomyelitis/chronic fatigue syndrome, long-COVID (for example, caused by an infection with a delta variant or an omicron variant of SARS-CoV-2 i) or chronic fatigue syndrome.

### Cardiovascular and Cerebrovascular Diseases

According to one embodiment of the invention, there is provided a composition of the invention for use in the treatment of cardiovascular and cerebrovascular diseases. For example, cardiovascular and cerebrovascular diseases selected from transient ischemia, hypertension, anti-platelet aggregation, formation of the internal carotid artery onset plate, Cerebral atherosclerosis, arterial atherosclerosis, infarction, myocarditis, meningitis, cerebral arteriosclerosis, hyperlipidaemia and basilar artery insufficiency. In a further embodiment, the cardiovascular and cerebrovascular diseases are selected from cerebral ischemia, cerebral thrombosis, cerebral embolism, cerebral stroke, hypertension and coronary heart disease, myocardial ischemia, arrhythmia, heart failure and angina pectoris.

### Metabolic Diseases

According to one embodiment of the invention, there is provided a composition of the invention for use in the treatment of metabolic diseases. For example, wherein the metabolic diseases are selected from: diabetes mellitus, obesity, metabolic syndrome, reduced metabolic rate, fatty liver disease (for example NAFLD and NASH), and decline in liver function. In a further embodiment composition of the invention are useful for weight management.

### Neurological Disorders

According to one embodiment of the invention, there is provided a composition of the invention for use in the treatment of neurological disorders, including neurodegenerative disorders. Examples of neurological disorders include: cognitive decline, memory decline, anxiety, depression, epilepsy, stroke, amyotrophic lateral sclerosis, dementia (e.g. vascular dementia and Alzheimer's disease), Parkinson's disease, and Huntington disease.

According to a further embodiment of the invention, there is provided a composition of the invention, for use in the prophylaxis or treatment of a disease state initiated or characterized (i) by a decline in cognitive function; or (ii) by mood disturbances. Such disease states can include, without limitation, neurodegenerative disease, cognitive disorder, mood disorder and stress and/or anxiety disorder.

According to a further embodiment, there is provided a composition of the invention, for use protecting against brain oxidative stress and/or reducing brain inflammation

### Cancer

According to one embodiment of the invention, there is provided a composition of the invention for use in the treatment of cancer. According to a further embodiment of the invention, there is provided a composition of the invention for inhibiting metastasis.

Examples of suitable cancers include: bladder cancer, B-cell lymphoma such as Hodgkin's lymphoma, T-cell lymphoma, T-cell acute lymphoblastic leukaemia, acute lymphoblastic leukaemia, chrome lymphoblastic leukaemia, acute myelogenous leukaemia, chronic myelogenous leukaemia, erythroleukemia, triple negative breast cancer, breast cancer, ovarian cancer, melanoma including paediatric melanoma, lung cancer such as squamous cell lung carcinoma and non small-cell lung cancer, pancreatic cancer, glioblastoma, colorectal cancer, head and neck cancer such a head and neck squamous cell carcinoma, cervical cancer, prostate cancer, liver cancer, oral squamous cell carcinoma, skin cancer, medulloblastoma, hepatocellular carcinoma, intrahepatic and extrahepatic cholangiocarcinoma, desmoid tumours, soft tissue sarcoma, adenoid cystic carcinoma, urothelial cancer, renal cancer, hepatocellular cancer, skin cancer, such as Merkel cell carcinoma, gastric cancer and gastroesophageal cancer.

### Muscle Pathological Conditions

Compositions of the invention find use in the treatment of muscle-related pathological conditions. Therefore, according to a further embodiment of the invention, there is provided a composition of the invention for use in the treatment or prevention of muscle pathological conditions. Muscle-related conditions include both conditions impacting generally healthy individuals as well as pathological conditions. Such muscle conditions found in healthy people or people affected by a disease include musculoskeletal diseases or disorders; cachexia; muscle wasting; age related decline in muscle function; pre-frailty; frailty; myopathies; neuromuscular diseases, such as Duchenne muscular dystrophy and other dystrophies; age-related sarcopenia; acute sarcopenia; muscle atrophy and/or cachexia, for example muscle atrophy and/or cachexia associated with burns, bed rest, limb immobilization, or major surgery, including thoracic, abdominal, and/or orthopedic surgery; and muscle degenerative disease.

Examples of age-related conditions that may be treated with compositions of the invention include sarcopenia and muscle wasting.

The myopathy may also be caused by a muscular dystrophy syndrome, such as Duchenne.

### Aging/Healthspan

According to a further embodiment of the invention, there is provided a composition of the invention for use in the increasing or prolonging of healthspan. Healthspan can be defined as the part of a person's life during which they are generally in good health. In a further embodiment of the invention, there is provided a composition of the invention, for slowing aging.

### Immune Health

In a further embodiment of the invention there is provided the use of a composition of the invention, for supporting and/or enhancing immune function.

According to a further aspect of the invention there is provided a composition of the invention, for use in a method of (i) raising an immune response to an antigen and/or (ii) enhancing, modulating or augmenting an immune response to an antigen in a human or animal subject.

According to a further aspect of the invention there is provided a composition of the invention, for use in a method of preventing, reducing or slowing inflammaging in a human or animal subject.

In a further embodiment of the invention there is provided the use of a composition of the invention, for use as an anti-inflammatory agent.

According to a further aspect of the invention there is provided a composition of the invention, for use in a method of treating immunosenescence.

According to a further aspect of the invention there is provided a composition of the invention, for use in a method of preventing, reducing or slowing stem cell senescence in a human or animal subject.

In a further embodiment of the invention there is provided the use of a composition of the invention, for supporting and/or enhancing immune function during or after cancer treatment.

In a further embodiment of the invention there is provided the use of a composition of the invention, for supporting and/or enhancing immune function remission after cancer treatment.

In a further embodiment of the invention there is provided the use of a composition of the invention, or supporting and/or enhancing immune function during and after hospital admissions.

### Skin

A composition of the invention for use in the treatment of a skin condition, disease or disorder. Examples of skin diseases, disorders or conditions are selected from the group consisting of melasma, chloasma, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome.

In a further embodiment of the invention, skin diseases, disorders and conditions include:
(a) skin aging,
(b) age spots;
(c) liver spots;
(d) dry skin;
(e) radiation induced skin damage (for example, IR, UV, alpha particles, beta particles of gamma irradiation);
(f) lentigo;
(g) hyperpigmentation, for example age-related hyperpigmentation of the skin, or post-inflammatory hyperpigmentation;
(h) melasma (chloasma/mask of pregnancy);
(i) skin irritation, for example, dermatitis;
(j) skin infection, for example, warts;
(k) inflammatory skin conditions (for example, atopic eczema, seborrhoeic eczema, polymorphous photodermatosis, psoriasis, vitiligo);
(l) uneven skin colour (smoothing out thereof);
(m) fine lines and/or wrinkles;
(n) linea nigra;
(o) melanosis;
(p) endocrine diseases, such as Addison's and Cushing's syndrome.

### Hair

In further embodiments of the invention, there is provided a composition of the invention, for use for treating or preventing hair diseases disorders or conditions.

In one embodiment, hair diseases, disorders and conditions are selected from denutrition-based alopecia, endocrine disorder-based alopecia, vascular disorder-based alopecia, alopecia premature, traction alopecia, alopecia areata, alopecia neurotica, pityriasis alopecia, Trichotillomania, alopecia maligna, female pattern alopecia, male pattern alopecia, androgenetic alopecia, telogen effluvium, tinea capitis, alopecia totalis hypotrichosis, genetic hypotrichosis simplex, systemic drug for alopecia-based hair-loss, mechanical hair-loss, traumatic alopecia, pressure alopecia, anagen effluvium, pityriasis alopecia, alopecia syphilitica, alopecia seborrheic, symptomatic alopecia, alopecia cicatrisata, and alopecia congenita. However, the hair-loss should be understood as meaning including all symptoms classified as the alopecia in this field, regardless of the direct or indirect cause of the occurrence of the hair-loss.

According to a further aspect of the invention there is provided a composition of the invention, for the treatment of hair loss, wherein the hair loss is caused by one or more of the following: skin disorders, a medicine, a disease, autoimmunity, iron deficiency, severe stress, scalp radiation, pregnancy or pulling at your own hair.

### Treatments:

The composition of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

### Non-Therapeutic Uses

### Muscle Function, Performance, Endurance

The composition finds use in the management normal physiological function in healthy individuals of conditions characterised by poor physical performance, impaired endurance capacity, and impaired muscle function. Compositions of the invention may improve physical performance in individuals with a disease, including young and elderly individuals. Compositions of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Compositions of the invention further provide for the improvement of endurance capacity. The endurance capacity refers to the time to fatigue when exercising at a constant workload, generally at an intensity <80% VO₂max. Compositions of the invention may improve endurance capacity in individuals with a disease, including young and elderly individuals. Compositions of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a composition to improve, maintain or reduce the loss of muscle function. Compositions of the invention may improve, maintain or reduce the loss of muscle function in individuals with a disease, including young and elderly individuals. Compositions of the invention may improve, maintain or reduce the loss of muscle function in healthy individuals, including athletes, non-athletic individuals, sedentary individuals and the elderly. For example, compositions of the invention may increase muscle strength as evidenced by the improvement of performing a physical activity, such as an exercise, for example, increased ability to lift weights or increased hand grip strength. Also, compositions of the invention may improve muscle structure, for example by increasing or maintaining muscle mass in conditions of normal muscle function, declining muscle function or impaired muscle function.

This invention further provides a composition to improve the physical performance or endurance capacity as perceived by the individual. For example, by the reduction of in perceived exertion or effort during exercise or an activity as determined using a self-reported questionnaire.

### Muscle Performance

The composition of the invention is useful in enhancing muscle performance. The invention thus provides a composition of the invention for use in enhancing muscle performance. The invention also provides a method of enhancing muscle performance by administering to a subject an effective amount of a composition of the invention. Administration can be self-administration. The enhanced muscle performance may be one or more improved muscle function, improved muscle strength, improved muscle endurance and improved muscle recovery.

The composition of the invention can thus be used in a method of improving physical endurance (e.g., ability to perform a physical task such as exercise, physical labor, sports activities), inhibiting or retarding physical fatigue, enhancing working capacity and endurance, reducing muscle fatigue, enhancing cardiac and cardiovascular function.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The composition of the invention may be used in enhancing muscle performance by administering a composition of the invention to a subject who is frail or pre-frail.

Muscle performance may be sports performance, which is to say the ability of an athlete's muscles to perform when participating in sports activities. Therefore, in a further embodiment, there is provided a composition of the invention for enhancing sports performance. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

In a further embodiment, there is provided a composition of the invention for use in a method of enhancing physical performance in a subject, for example, in an elite athlete or sub-elite athlete.

According to a further embodiment of the invention, there is provided a composition of the invention, for use in a method of enhancing physical performance in a subject, for example, in an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

According to a further embodiment of the invention, there is provided a composition of the invention for use in a method of enhancing physical recovery in a subject, for example, in an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUCCK). In further embodiments, enhancing recovery comprises lowering C-reactive protein (CRP) levels in the subject, compared to baseline, following an aerobic activity as measured by area under the plasma concentration-time curve (AUCCRP).

### Immune Function

In a further embodiment of the invention there is provided the use of a composition of the invention, for maintaining or enhancing immune health.

In a further embodiment of the invention there is provided the use of a composition of the invention, for reducing or slowing inflammaging.

In a further embodiment of the invention there is provided the use of a composition of the invention, for slowing immune aging.

In a further embodiment of the invention there is provided the use of a composition of the invention, for reducing immune cell aging.

In a further embodiment of the invention there is provided the use of a composition of the invention, for boosting immune function.

In a further embodiment of the invention there is provided the use of a composition of the invention, for supporting a healthy immune system.

In a further embodiment of the invention there is provided the use of a composition of the invention, for prevention or management of colds and/or flu.

In a further embodiment of the invention there is provided the use of a composition of the invention, for the treatment of infection.

In a further embodiment of the invention there is provided the use of a composition of the invention, for improving vaccination response.

In a further embodiment of the invention there is provided the use of a composition of the invention, for enhancing an immune response against infection.

In a further embodiment of the invention there is provided the use of a composition of the invention, for protecting against infection, for example, protection against colds and flu.

In a further embodiment of the invention there is provided the use of a composition of the invention, for immune support.

In a further embodiment of the invention there is provided the use of a composition of the invention, for reducing the risk of respiratory tract infections (for example, colds, flu or pneumonia).

In a further embodiment of the invention there is provided the use of a composition of the invention, for increasing the rate of recovery from respiratory tract infections (for example, colds, flu or pneumonia).

In a further embodiment of the invention there is provided the use of a composition of the invention, for increasing CD8 positive T-cells, for example, naïve CD8-positive T-cells.

### Brain Function

According to a further embodiment of the invention there is provided the use of a composition of the invention, for maintaining or enhancing brain health.

According to a further embodiment of the invention there is provided the use of a composition of the invention, for promoting healthy brain function.

According to a further embodiment, there is provided a composition of the invention, for use for optimising brain health.

According to a further embodiment, there is provided a composition of the invention, for use to improve, protect, and maintain brain function and cognition.

According to a further embodiment, there is provided a composition of the invention, for use for enhancing mental alertness, for example, mental accuracy and clearer/sharper thinking.

According to a further embodiment, there is provided a composition of the invention, for use for increasing psychomotor speed and/or reaction time.

According to a further embodiment, there is provided a composition of the invention, for use for enhancing/improving memory.

According to a further embodiment, there is provided a composition of the invention, for use for enhancing/improving learning.

According to a further embodiment, there is provided a composition of the invention, for use for enhancing/improving reasoning, for example, promoting mental processing.

According to a further embodiment, there is provided a composition of the invention, for use for enhancing/improving focus, for example, concentration, complex attention and/or sustained attention.

According to a further embodiment, there is provided the use of a composition of the invention, for stress relief and/or anxiety relief.

According to a further embodiment, there is provided the use of a composition of the invention, for treating stress and/or anxiety.

According to a further embodiment, there is provided the use of a composition of the invention, for treating depression.

According to a further embodiment, there is provided the use of a composition of the invention, for improving mood.

According to a further embodiment, there is provided the use of a composition of the invention, for enhancing sleep and/or relaxation.

According to a further embodiment, there is provided the use of a composition of the invention, for improving cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

According to a further embodiment, there is provided a composition of the invention, for use to improve, protect, and maintain brain function and cognition.

### Skin

According to a further embodiment, there is provided the use of a composition of the invention, for treating or preventing skin conditions in generally healthy subjects. For example, it is suitable for use by subjects with skin prone to the ailments mentioned herein, for example subjects with intermittent skin issues, for example, skin prone to sunburn, prone to acne and prone to inflammation.

According to a further embodiment of the invention, there is provided use of a composition of the invention, for one or more of the following:
a) maintaining or enhancing skin health and appearance;
b) for maintaining or enhancing skin energy;
c) for maintaining or enhancing skin collagen;
d) for maintaining or enhancing skin elasticity
e) reducing skin biological aging;
f) supporting healthy skin aging;
g) reducing skin wrinkles, and/or fine lines;
h) improving skin mitochondrial function;
i) maintaining or improving skin hydration;
j) promoting replenishment of skin ceramide levels;
k) improving barrier function;
l) protecting against free radical damage; and/or
m) improving skin dryness;
n) improving photodamaged skin;
o) Improving skin health in skin prone to redness, inflammation, acne or dryness
p) helping replenish ceramide stores; and
q) reducing skin redness and irritation.

In a further embodiment of the invention, there is provided a composition of the invention, for one or more of the following uses:
(a) skin bleaching and/or lightening skin colour and/or lightening skin tone;
(b) skin whitening;
(c) protection of skin caused by damage by the environment (For example, damage caused by sunlight/UV irradiation and/or damage caused by pollution);
(d) decreasing pigmentation; and
(e) suppressing melanin production.

In a further embodiment of the invention, there is provided a composition of the invention, for one or more of the following uses:
(a) renewal and/or revitalization of skin appearance;
(b) enhancement of skin energy supply;
(c) improvement in skin texture;
(d) enhancement of skin radiance;
(e) skin rejuvenation/regeneration;
(f) reduction of pore and micro line visibility;
(g) improvement in skin respiration.
(h) support optimal cellular function in skin;
(i) promote/activate skin detoxifying processes;
(j) promote collagen repair, and/or
(k) improve skin stem cell health.

### Hair

According to a further aspect of the invention there is provided a composition of the invention, for stimulating hair growth.

According to a further aspect of the invention there is provided a composition of the invention for the treatment of hair loss

According to a further aspect of the invention there is provided a composition of the invention, for preventing or ameliorating hair loss.

According to a further aspect of the invention there is a composition of the invention, for delaying the onset of hair loss.

According to a further aspect of the invention there is provided a composition of the invention, for treating hair thinning.

According to a further aspect of the invention there is provided a composition of the invention, for use in a method of increasing hair thickness (i.e. number of hair fibres per surface area) and/or number hair follicles actively producing hair fibres.

According to a further aspect of the invention there is provided a composition of the invention, for slowing or preventing premature greying of hair.

In a further embodiment of the invention, there is provided a composition of the invention, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) promoting new hair growth;
(h) hair maintenance;
(i) scalp health improvement,
(j) improving or maintaining hair strength,
(k) or improving survival of hair transplants; and/or
(l) hair growth restoration promotion.

In a further embodiment of the invention, there is provided a composition of the invention, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

### Nails

Compositions of the invention find use in the treatment or prevention of diseases, disorders and conditions associated with nails. Such uses in diseases, disorders and conditions include use in both pathological and non-pathological conditions and cosmetic indications.

In a further embodiment of the invention, nail diseases, disorders and conditions include nail deformities (changes in nail shape) and nail dystrophies (changes in nail texture, colour or both.

Nail conditions include: brittle nails, triangular worn down nails, trachyonychia, and habit tic deformity.

Therefore, according to a further embodiment of the invention, there is provided a composition of the invention, for use in the treatment or prevention of diseases, disorders and conditions associated with nails, for example, nail deformities (changes in nail shape) and nail dystrophies (changes in nail texture, colour or both and brittle nails, triangular worn down nails, trachyonychia, and habit-tic deformity.

### Compositions

The uses and methods of the present invention preferably involve oral administration of the compositions of the invention. Any suitable oral composition may be used. Accordingly, the use of a range of compositions which are suitable for oral administration, is envisaged. Thus, in some embodiments, the composition is administered in the form of an oral composition and one or more excipients suitable for oral administration. Oral compositions may comprise compositions having the form of a pill, tablet, capsule, caplet, lozenge, pastille, granules, powder for suspension, oral solution, oral suspension, oral emulsion, syrup, or the like.

In a further embodiment of the invention, the composition is administered by any means known to the skilled person for administration such as, intramuscular, sublingual, cutaneous, inhalation and auricular. Oral administration is preferred.

Compositions may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In some preferred embodiments, the use or method comprises administration of the compound of formula (I) or salt thereof (e.g. urolithin A), in micronized form. Micronization enables the compound of formula (I) to disperse or dissolve more rapidly. Micronisation can be achieved by methods established in the art, for example compressive force milling, hamermilling, universal or pin milling, or jet milling (for example spiral jet milling or fluidised-bed jet milling) may be used. Jet milling is especially suitable. If micronized compound is used, then preferably the compound has a Do size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm. Preferably, the compound has a Do size of under 100 μm. More preferably, the compound has a D₉₀ size of under 75 μm, for example under 50 μm, for example under 25 μm, for example under 20 μm, for example under 15 μm. The compound preferably has a D₉₀ in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a D₉₀ in the range 8.2 to 16.0 μm, a D₅₀ in the range 2.8 to 5.5 μm and a D₁₀ in the range 0.5 to 1.0 μm.

In a further embodiment, the compound of formula (I) or salt thereof has a size distribution selected from one of the following:
(i) D₅₀ size in the range 0.5 to 50 μm and a D₉₀ size in the range 5 to 100 μm,
(ii) the compound has a D₉₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a D₅₀ size under 50 μm and a D₉₀ size under 75 μm;
(v) the compound of Formula (I) has a D₅₀ size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉₀ size under 15 μm; or
(vi) the compound of Formula (I) has a D₅₀ size of 10 μm and a Do size of 20 μm.

In one embodiment, the composition is administered in the form of a composition comprising: a) a medium-chain triglyceride; b) the compound of formula (I) or salt thereof; and ginsenosides and/or notoginsenosides. Within those embodiments, preferably the compound of formula (I) (e.g. urolithin A) is in micronized form. Compositions comprising a urolithin or salt thereof, and a medium chain triglyceride can be found in International patent application: WO 2017/036992.

For the avoidance of doubt, compounds in compositions of the invention may be formulated in the same composition or formulated in separate compositions for simultaneous, separate or sequential administration.

### Kits

Also within the scope of the present invention are kits, comprising composition of the invention. Kits typically include a label indicating the intended use of the contents of the kit and instructions for use. The term “label” includes any writing, or recorded material supplied on or with the kit, or which otherwise accompanies the kit.

The invention further provides kits comprising:
(a) a compound of formula (I), or a salt, prodrug, metabolite or derivative thereof, as defined in claim 1;
(b) one or more ginseng-derived compounds or a ginseng extract; and/or one of more notoginseng-derived compounds or a notoginseng extract;
(c) a container, or containers, for containing said agents; and
(d) optionally instructions for simultaneous, separate or sequential administration.

In a further embodiment, the invention provides a kit comprising:
(a) about 100 mg to about 2000 mg of a compound of formula (I), or a salt, prodrug, metabolite or derivative thereof;
(b) about 1 mg to about 50 mg, for example, about 5 mg to about 30 mg, ginsenosides and/or notoginsenosides.
(c) a container, or containers, for containing said agents; and
(d) optionally instructions for simultaneous, separate or sequential administration.

### Preparation of Compositions of the Invention

In a further embodiment, there is provided a process for preparing a composition of the invention, which comprises, mixing
(a) a compound of formula (I), or a salt, prodrug, metabolite or derivative thereof, for example, urolithin A;
(b) one or more ginseng-derived compounds or a ginseng extract; and/or one of more notoginseng-derived compounds or a notoginseng extract, and
(c) one or more excipients, for example, one of more excipients suitable for oral administration.

The term ‘about’ refers to a tolerance of ±20% of the relevant value, for example ±15% of the relevant value, such as ±10% of the relevant value or ±5% of the relevant value.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing

The term “ginseng” refers to Panax ginseng.

Compositions of the invention also include compounds or extracts from other species of the genus Panax of the family Araliaceae. These include P. japonicum, P. quinquefolium (American ginseng), P. trifolium, and P. pseudoginseng.

The term ‘Notoginseng’ refers to Panax notoginseng.

The term “pharmaceutically acceptable” means approved by a regulatory agency of the Federal or a state government or listed in the U.S. Pharmacopeia or other generally recognized pharmacopeia for use in animals, and more particularly in humans.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a patient from non-fixed (separate) dosage forms in separate actions. The administration actions may, or may not, be linked by a specified time interval. For example, administering compounds over a specified time such as once every 14 to 21 days.

The term “simultaneous” administration means the administration of each of two or more compounds to a patient in a single action such as where each compound are administered independently at substantially the same time or separately within time intervals that allow the compounds to show a cooperative therapeutic effect.

The term “therapeutically effective amount” as used herein refers to the amount of a compound or compounds that, when administered, is sufficient to prevent the development of, or to relieve to some degree, one or more symptoms of the disease that it targets. The particular dose of each compound administered according to this invention will of course be determined by the particular conditions surrounding the case, including the compound administered, the route of administration, the particular condition being treated, as well as considerations such as age, weight and sex of the treated subject.

### EXAMPLES

The invention will now be illustrated with respect to the following non-limiting examples.

### Example 1a: Representative Powder or Gel Composition

| Composition | Per 100 g | |
|---|---|---|
| Protein | 10-80 | g |
| Carbohydrates | 20-40 | g |
| Fat | 0-20 | g |
| Polyunsaturated Fatty Acids | 0-5 | g |
| Fiber | 0-5 | g |
| Vitamins | 0-100% of respective RDAs | |
| Minerals | 0-100% of respective RDAs | |
| Folic Acid | 0-1000 | μg |
| Niacin | 0-100 | mg |
| Creatine | 0-20 | g |
| Ginsenoside Rg1 | 0.025-5 | g |
| Urolithin A | 0.025-5 | g |

### Example 1 b: Representative Powder or Gel Composition

| Composition | Per 100 g | |
|---|---|---|
| Protein | 10-80 | g |
| Carbohydrates | 20-40 | g |
| Fat | 0-20 | g |
| Polyunsaturated Fatty Acids | 0-5 | g |
| Fiber | 0-5 | g |
| Vitamins | 0-100% of respective RDAs | |
| Minerals | 0-100% of respective RDAs | |
| Folic Acid | 0-1000 | μg |
| Niacin | 0-100 | mg |
| Creatine | 0-20 | g |
| Ginsenoside Rb1 | 0.025-5 | g |
| Urolithin A | 0.025-5 | g |

### Example 1c: Representative Powder or Gel Composition

| Composition | Per 100 g | |
|---|---|---|
| Protein | 10-80 | g |
| Carbohydrates | 20-40 | g |
| Fat | 0-20 | g |
| Polyunsaturated Fatty Acids | 0-5 | g |
| Fiber | 0-5 | g |
| Vitamins | 0-100% of respective RDAs | |
| Minerals | 0-100% of respective RDAs | |
| Folic Acid | 0-1000 | μg |
| Niacin | 0-100 | mg |
| Creatine | 0-20 | g |
| Ginsenoside Rg1 | 0.025-5 | g |
| Ginsenoside Rb1 | 0.025-5 | g |
| Urolithin A | 0.025-5 | g |

### Example 2: Representative Skin Face Cream

| Composition | Per 100 g |
|---|---|
| Protein | 10 g-80 g |
| Carbohydrates | 20 g-40 g |
| Fat | 0 g-20 g |
| Polyunsaturated Fatty Acids | 0 g-5 g |
| Fiber | 0 g-5 g |
| Vitamins | 0-100% of respective RDAs |
| Minerals | 0-100% of respective RDAs |
| Folic Acid | 0 μg-1000 μg |
| Niacin | 0 μg-100 mg |
| Creatine | 0 g-20 g |
| Ginsenoside Rg1 | 0.025 g-5 g |
| Urolithin A | 0.025 g-5 g |

### Example 3: Representative Face Cream or Cosmetic Composition

| Composition comprising | % of Cream (w/w) |
|---|---|
| AQUA (WATER) | |
| GLYCERIN | 2% to 8% |
| BUTYLENE GLYCOL | 1 to 7% |
| DIGLYCERIN | 2 to 5% |
| NIACINAMIDE | 2 to 5% |
| SQUALANE | 0.5 to 3% |
| UROLITHIN A | 1% to 5% |
| CAFFEINE | 0.2% to 1% |
| HYDROXYACETOPHENONE | 0.2% to 1% |
| PANTHENOL | 0.2 to 1% |
| TREHALOSE | 0.1 to 1% |
| Ginesenoside(s), for example, Rg1 | 1 to 7 |

### Example 4: Representative Face Cream or Cosmetic Composition

| Composition comprising | % of Cream (w/w) |
|---|---|
| AQUA (WATER) | |
| GLYCERIN | 2% to 8% |
| BUTYLENE GLYCOL | 1 to 7% |
| DIGLYCERIN | 2 to 5% |
| NIACINAMIDE | 2 to 5% |
| SQUALANE | 0.5 to 3% |
| UROLITHIN A | 1% to 5% |
| CAFFEINE | 0.2% to 1% |
| HYDROXYACETOPHENONE | 0.2% to 1% |
| PANTHENOL | 0.2 to 1% |
| TREHALOSE | 0.1 to 1% |
| Ginseng extract | 10% to 30% |

### Example 5: Effects of Ginsenoside Rg1 and Urolithin A (UA) on IL-6 Levels and Inflammation

C2C12 Myoblasts purchased from ATCC (ref. CRL-1772) were seeded at 8000 cells per well in a 96 well plate (Greiner) using a DMEM Glutamax 4.5g D-glucose (Gibco, ref 31966021) supplemented with FIBS 10% (PanBiotech, ref P190902), 1% PenStrep (Biowest) and 1% Hepes (Biowest). Cells were then differentiated for 6 days using a differentiation medium (DMEM Glutamax 4.5g D-glucose (Gibco, ref 31966021), 2% Horse serum (Thermofisher, ref 160501229) 1% PenStrep and 1% Hepes). All media were sterilized with a 0.45 um filter unit (VWR). After 4 days of the differentiation cells were treated for 24 h with 1) Urolithin A (UA) resuspended in DMSO, 2) ginsenoside Rg1 (MedChemExpress, HY-N0045/CS-3832) or 3) a combination of the two at the indicated doses. A cytokine mixture (“cytomix”) was added at day 5 in addition of the previous treatment for another 24 h, cytokines were respectively used at 5ng (mIFN-γ (Roche ref. 11276905001), TNF-α (Peprotech ref. 300-01A) and IL-1β (Proteintech ref. 200-01B)). The medium was collected and an ELISA for Interleukin-6 were performed following the instruction provided by the supplier (Proteintech ref. KE10007). In particular, the IL-6 ELISA kit is to be used to detect and quantify protein levels of endogenous mouse IL-6. The assay recognizes mouse IL-6. An antibody specific for mouse IL-6 has been pre-coated onto the microwells. An antibody specific for mouse IL-6 has been pre-coated onto the microwells. The IL-6 protein in samples is captured by the coated antibody after incubation. Following extensive washing, another antibody of biotinylated specific for mouse IL-6 is added to detect the captured mouse IL-6 protein. For signal development, Streptavidin-HRP is added, followed by Tetramethyl-benzidine (TMB) reagent. Solution containing sulfuric acid is used to stop colour development and the colour intensity which is proportional to the quantity of bound protein is measurable at 450 nm with the correction wavelength set at 630 nm, using a microplate plate reader (Fluostar Optima, BMG Labtech).

### Results

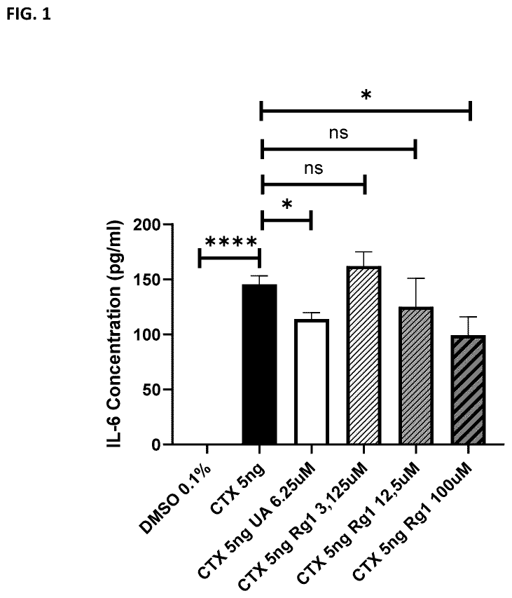

We exposed of C2C12 to a mix of pro-inflammatory cytokines (cytomix, CTX) to induce inflammation signals. Interleukin-6 (IL-6), a multifunctional cytokine produced in cells and induced by a variety of stimulating signals. IL-6 production and release in the media is a proxy of the inflammation induced by treatment with the cytokines part of the cytomix (Tosato & Jones (1990) Blood 75(6), 1305-10). With cytomix treatment, we observed a significant increase in Interleukin 6 (IL-6) secretion, measured as the concentration of IL-6 in the cell culture media. Urolithin A pre-treatment for 24 hours at a dose of 6.25 μM significantly decreased IL-6 secretion (FIG. 1). Ginsenoside Rg1 pre-treatment for 24 hours led to a dose dependent decrease in IL-6, with no effects at the lower 3.125 μM dose, a non-significant trend in reduction at the 12.5 μM dose and a significant decrease at the highest 100 μM dose (FIG. 1).

Given the two different mechanisms of action of UA and Rg1, we explored potential synergistic effect of combined treatment with UA at a dose of 6.25 μM and either Rg1 at either 3.125 μM, and 100 μM.

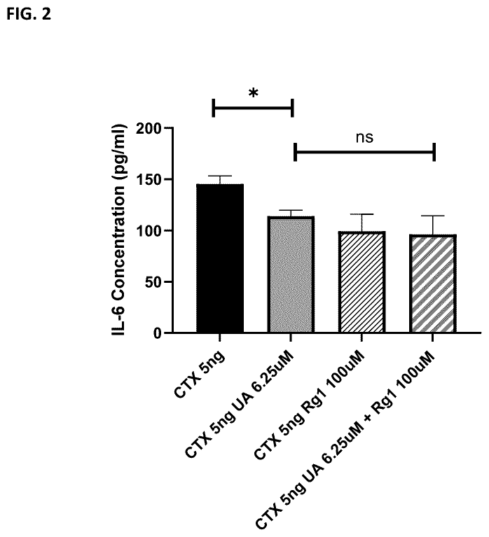

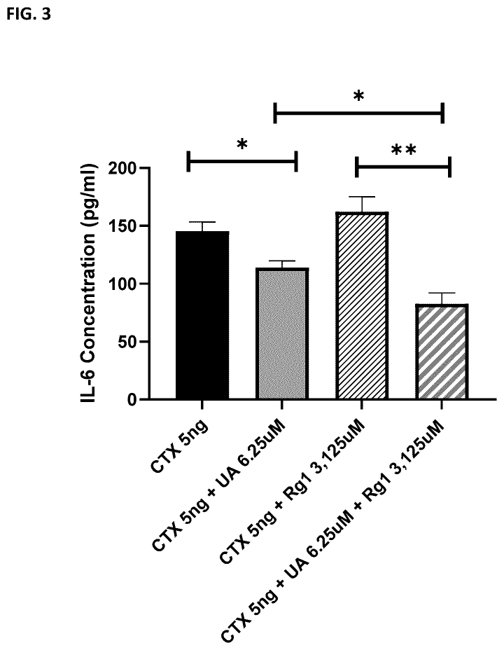

Compared to treatments with individual compounds, there was no significant further decline in inflammation when combining UA with the higher dose Rg1 (100 μM) (FIG. 2). Surprisingly, while treatment with the 3.125 μM low dose of Rg1 did not show any anti-inflammatory effect alone, its co-treatment with UA led to synergist effects on the IL-6 inflammation marker (FIG. 3 and table 1).

Ginsenosides such as Rg1 have been shown to reduce inflammation in certain cell-based models. For instance, they reduced high glucose induced inflammation after 48 treatment at doses from 2.5 μM to 10 μM (Liu et al (2021) Ann Transl Med 9(24), 1789). Here, we confirmed the anti-inflammatory effects of Rg1. As expected, the shorter treatment duration compared to literature above (24 hours vs 48 hours) led to smaller effects and no effects at the lower dose of 3.125 μM. However, this shorter treatment allowed us to identify an unexpected time window and dosing in which UA unlocks and boosts the effects of low doses of Rg1, that alone would otherwise have no effects.

TABLE 1

| | | % | |
|---|---|---|---|
| | | reduction | Adjusted |
| Tukey's multiple comparisons test | Summary | IL-6 | P Value |
| CTX 5 ng vs. UA 6.25 μM + CTX | * | −16% | 0.0187 |
| CTX 5 ng vs. Rg1 3.125 μM + CTX | ns | +9.8% | 0.2776 |
| CTX 5 ng vs. UA 6.25 μM + | * | −38% | 0.0337 |
| Rg1 3.125 μM + CTX | | | |
| Rg1 3.125 μM + CTX | ** | −55% | 0.0014 |
| vs. UA 6.25 μM + | | | |
| Rg1 3.125 μM + CTX | | | |

### Equivalents

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

1. A composition comprising:
(a) a compound of formula (I), or a salt, prodrug, metabolite or derivative thereof; 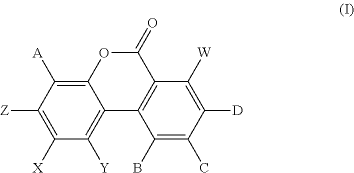
wherein:
A, B, C, D, W, X, Y and Z are each independently selected from H and OH; and
(b) one or more ginseng-derived compounds or a ginseng extract; and/or
one of more notoginseng-derived compounds or a notoginseng extract.

2. The composition as claimed in claim 1, comprising:
(a) a compound of formula (I), or a salt thereof; and
(b) one or more ginseng-derived compounds or a ginseng extract.

3. The composition as claimed in claim 1, comprising:
(a) a compound of formula (I), or a salt thereof; and
(b) one of more notoginseng-derived compounds or a notoginseng extract.

4. The composition as claimed in claim 1, wherein the ginseng extract is derived from red ginseng or white ginseng, for example, fermented red ginseng or fermented white ginseng.

5. The composition as claimed in claim 1, wherein the ginseng-derived compound comprises one or more gintonins.

6. The composition as claimed in claim 1, wherein the ginseng-derived compound comprises one or more ginsenosides.

7. The composition as claimed in claim 6, wherein the ginsenosides comprise dammarane-type saponins, (for example, protopanaxadiol-type saponins and protopanaxatriol-type saponins), ocotillol-type saponins, and/or oleanane-type sapoinins.

8. The composition as claimed in claim 1, wherein the notoginseng-derived compound comprises one or more notoginsenosides.

9. The composition as claimed in claim 8, wherein the notoginsenosides comprise dammarane-type saponins, (for example, protopanaxadiol-type saponins and protopanaxatriol-type saponins) and/or ocotillol-type saponins.

10. The composition, as claimed in claim 6, wherein the ginsenosides and/or notoginsenosides comprise one or more selected from Rb1, Rd, Re, Rg1, Rg2, Rh1, and gypenoside XVII.

11. The composition as claimed in claim 10, wherein the ginsenosides and/or notoginsenosides comprise one or more selected from Rg1 and Rb1, for example, Rg1.

12. The composition as claimed in claim 6, wherein the ginsenosides are one or more selected from Rb1, Rb2, Rg3, Rh2, Rh3, Rg1, Rg2, and Rh1.

13. The composition as claimed in claim 8, wherein the notoginsenosides are one or more selected from R1, Rt, R2, R3, R4, and R6.

14. The composition as claimed in claim 1, wherein the compound of formula (I) is selected from urolithin A, urolithin B, urolithin C and/or urolithin D, for example, urolithin A.

15. The composition as claimed in claim 1, wherein the composition comprises one or more further active agents.

16. The composition as claimed in claim 1, wherein the composition is administered at a dose of about 4.5-18 mg/kg of a compound of formula (I).

17. The composition as claimed in claim 6, wherein the ginsenosides and/or notoginsenosides are administered at a dose of about 0.01 mg/kg to about 0.625 mg/kg ginsenosides and/or notoginsenosides

18. The composition as claimed in claim 1, comprising:
(a) 100 to 2000 mg of a compound of formula (I), or a salt, prodrug, metabolite or derivative thereof, for example, urolithin A; and
(b) about 1 mg to about 50 mg, for example, about 5 mg to about 30 mg, ginsenosides or notoginsenosides.

19. (canceled)

20. (canceled)

21. A method of improving muscle function, improving immune function, improving brain function, improving skin health, and/or improving hair health comprising administering to a subject in need thereof an effective amount of a composition as claimed in claim 1.

22. (canceled)

23. (canceled)

24. A kit comprising:
(a) a compound of formula (I), or a salt, prodrug, metabolite or derivative thereof, as defined in claim 1;
(b) one or more ginseng-derived compounds or a ginseng extract; and/or
one of more notoginseng-derived compounds or a notoginseng extract;
(c) a container, or containers, for containing said agents; and
(d) optionally instructions for simultaneous, separate or sequential administration.

25. (canceled)
